(19) United States
(12) Patent Application Publication
(10) Pub. No.: US 2023/0174667 A1
(43) Pub. Date: Jun. 8, 2023

(54) INHIBITION OF SARS-COV-2 INFECTION THROUGH SYNDECANS

(71) Applicant: Pharmacoidea Kft, Szeged (HU)

(72) Inventors: Anett HUDÁK, Szarvas (HU); Tamás LETOHA, Szeged (HU)

(21) Appl. No.: 17/924,427

(22) PCT Filed: May 11, 2021
(86) PCT No.: PCT/HU2021/000006

§ 371 Date: Nov. 10, 2022

(30) Foreign Application Priority Data
May 12, 2020 (HU) P2000155

(51) Int. Cl. C07K 16/28; A61K 38/08; A61K 38/10; A61K 38/39; A61K 38/17; A61K 31/7105; A61K 31/407; A61K 31/55; A61K 31/727; A61P 31/14

(52) U.S. Cl. CPC C07K 16/2896; A61K 38/08; A61K 38/10; A61K 38/39; A61K 38/177; A61K 31/7105; A61K 31/407; A61K 31/55; A61K 31/727; A61P 31/14

(57) ABSTRACT

The invention relates to agents that inhibit the binding of SARS-CoV-2 to SDCs or the endocytosis of SARS-CoV-2 with SDCs for use in the treatment of SARS-CoV-2 infection by inhibiting the cellular entry of SARS-CoV-2 and/or the SARS-CoV-2-induced inflammatory response. The invention relates further on to a method of inhibiting SARS-CoV-2 infection, the cellular entry of SARS-CoV-2, and the SARS-CoV-2-induced inflammatory response comprising contacting the SARS-CoV-2 target cells with an agent that inhibits the binding of SARS-CoV-2 to SDC or the endocytosis of SARS-CoV-2 with SDCs. The agent as SDC-specific mono- or polyclonal antibody used may be a. an antibody specific for the human SDC isoforms (SDC1, -2, -3, -4), or b. as SDC-specific peptides, peptide ligands that interact with the extracellular domain of human SDC isoforms (SDC1, -2, -3, -4), such as TAT (amino acid sequence YGRKKRRQRRR), penetratin (amino acid sequence RQIKIWFQNRRMKW), polyarginine (amino acid sequence RRRRRRRR) and their analogs of at least four amino acids in length, synthesized from D-, L- or other amino acid derivatives, or peptides and peptidomimetics interacting with the glycosaminoglycan side chains of SDCs, containing the conserved heparin-binding motif, PRRAR, or analogs of at least four amino acids in length, synthesized from D-, L- or other amino acid derivatives or conjugates of the former with other active agents and carriers (liposomes and other polymeric nanoparticles); or c. as SDC specific low- and high-molecular-weight ligands, any agents capable of binding to the extra- and intracellular domains of SDC, including the glycosaminoglycan side chains of SDCs; or d. as agents derived from SDCs, recombinant proteins derived from the extra- and intracellular domains of human SDC isoforms (SDC1, -2, -3, -4) and analogs synthesized from D-, L- or other amino acid derivatives of at least 4 amino acids in length, and derivatives of glycosaminoglycan side chains and glycosaminoglycan analogs of human SDC isoforms (SDC1, -2, -3, -4); or e. as agents reducing the expression of SDCs, nucleic acid-based agents capable of reducing the expression of SDCs and their conjugates and complexes packaged in viral and non-viral carriers (liposomes and other polymeric nanoparticles); or f. as agents containing the conserved intracellular domain of SDCs peptides, peptidomimetics and recombinant proteins of at least four amino acids in length, synthesized from D-, L- or other amino acid derivatives containing the sequence of any conserved region of the intracellular domain of human SDC isoforms (SDC1, -2, -3, -4); or g. as an inhibitor of endocytosis of syndecans, low molecular weight agents that inhibit the interaction of SDCs with PKCa, the RAC1, and Wnt pathways.

The present invention relates to a method of inhibiting SARS-CoV-2 infection with syndecan derivatives and syndecan-targeting agents.

The severe acute respiratory syndrome coronavirus 2, SARS-CoV-2, is an emerging infectious human coronavirus causing COVID-19 [Zowalaty and Jarhult 2020 (One Health (9) 100124); Chu et al. 2020 (Clin Chem (66) 549-555)].

SARS-CoV-2 is a positive-sense, single-stranded RNA betacoronavirus [Mason 2020 (Eur Respir J (55) 2000607; Ye et al. 2020 (Int J Biol Sci (16), 1686-1697)] responsible for the WHO-declared COVID-19 pandemic [Zheng 2020 (Int J Biol Sci (16) 1678-1685)]. Specific antiviral agents against SARS-CoV-2 infection are currently lacking [Guo et al. (Mil Med Res (7) 11)].

Little is known about the cellular biology of SARS-CoV-2 infection. The main steps of SARS-CoV-2 infection are greatly postulated from previous studies with SARS-CoV, a coronavirus strain responsible for the first SARS outbreak in 2002-2003 [Mason 2020 (Eur Respir J (55) 2000607)]. According to the current scientific consensus, SARS-CoV-2 enters cells in a fashion similar to SARS-CoV. Namely, the angiotensin-converting enzyme 2 (ACE2) has been identified as the primary cell entry receptor for both SARS-CoV-2 and SARS-CoV [Hoffmann et al. 2020 (Cell (181) 271-280); Wan et al. 2020 (J Virol (94) pii: e00127-20.)]. However, scientific evidence shows that endocytosis of SARS-CoV also occurs through a novel, clathrin- and caveolae-independent endocytic pathway, mediated by attachment to cell surface heparan sulfate proteoglycans (HSPGs) [Lang et al. 2011 (PLoS One (6) e23710; Wang et al. 2008 (Cell Res (18), 290-301)]. However, the role of HSPGs in SARS-CoV-2 has not yet been fully elucidated.

Due to their diverse heparan sulfate (HS) side chains, HSPGs, one of the most populated families of cell surface membrane proteins, play essential roles in cell physiology by serving as receptors for many ligands (e.g., cytokines, growth factors, etc.) [Williams and Fuki 1997 (Curr. Opin. Lipidol 8(5), 253-262), Bishop, Schuksz et al. 2007 (Nature 446(7139) 1030-1037), Couchman 2010 (Annu. Rev. Cell Dev. Biol. 26, 89-114), Iozzo and Karamanos 2010 (FEBS J. 277(19), 3863)].

Two types of cell membrane HSPGs are found in mammalian cells: the central nervous system (CNS) expressed glypicans that are anchored to the cell membrane via glycosylphosphatidylinositol, and the ubiquitous transmembrane syndecans (SDCs) [Christianson and Belting 2014 (Matrix Biol. (35)) 51-55)]. Four isoforms of SDCs are known: syndecan-1 (SDC1) expressed on epithelial and plasma cells, syndecan-2 (SDC2) on endothelial cells and fibroblasts, syndecan-3 (SDC3) on neurons, and the universally expressed syndecan-4 (SDC4) [Tkachenko, Rhodes et al. 2005 (Circ. Res. 96(5), 488-500), Afratis, Nikitovic et al. 2017 (FEBS. J. 284(1), 27-41)].

SDCs are made of three main parts: the evolutionarily conserved transmembrane and intracellular domains and a more diverse extracellular domain (ectodomain) containing HS and chondroitin sulfate side chains [Tkachenko, Rhodes et al. 2005 (Circ. Res. 96 (5), 488-500)]. Due to their diverse HS side chains, SDCs serve as binding sites for many endogenous and exogenous ligands, including viruses. Through their evolutionarily conserved intracellular domains, SDCs interact with several intracellular signaling molecules [Tkachenko, Rhodes et al. 2005 (Circ. Res. 96 (5), 488-500); Christianson and Belting 2014 (Matrix Biol. (35) 51-55)]. SDCs play a central role in viral infection: binding of viruses to SDCs induces a yet not fully elucidated endocytic process that delivers the virus into the host cell [Bartlett & Park 2010 (Expert Rev Mol Med (12) e5)]. However, the role of SDCs in SARS-CoV-2 is not yet known.

In our experiments, we investigated the role of different SDC isoforms in the cellular uptake of SARS-CoV-2. Heat-inactivated SARS-CoV-2 (ATCC, cat. no. ATCC-VR-1986HK) and transgenic cell lines expressing different SDC isoforms were applied.

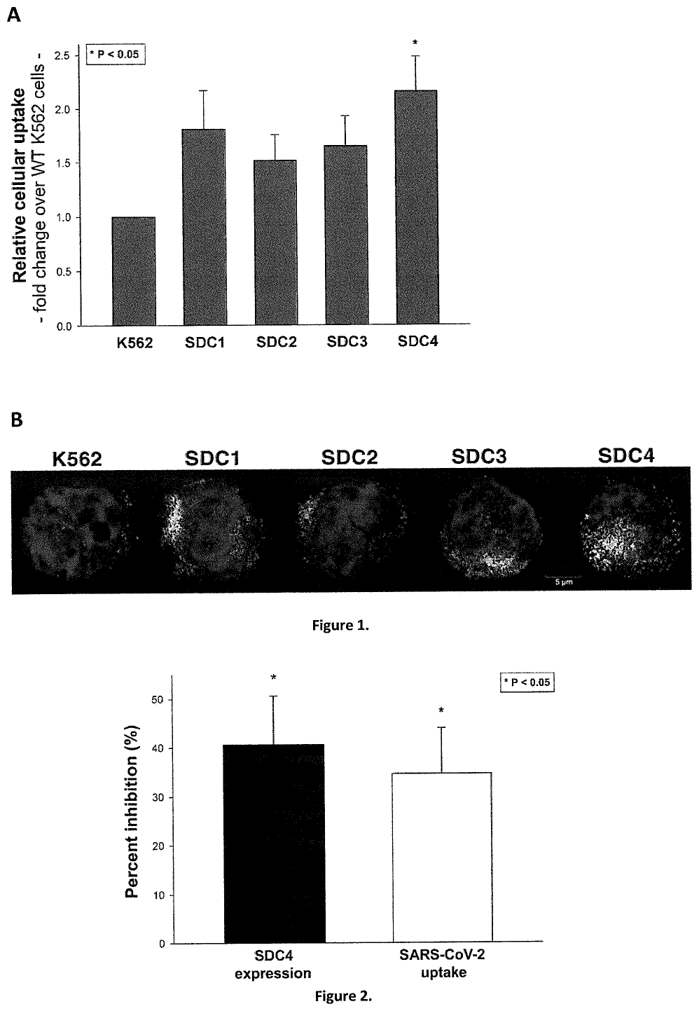

As wild-type (WT) K562 cells do not express HSPGs except for minimal betaglycan and SDC3 [Letoha et al. 2019 (Sci Rep (9) 1393)], K562 cells are ideal cellular models to study the effects of SDCs on SARS-CoV-2 entry. Therefore, WT K562 cells were transfected with the various SDC isoforms, and the resulting stable SDC transfectants were standardized according to their HS expression [Letoha et al. 2019 (Sci Rep (9) 1393)]. Thus SDC transfectants expressing equal amounts of HS, along with WT K562 cells were incubated with 5 MOI (MOI: multiplicity of infection per cell) heat-inactivated SARS-CoV-2 for 18 h at 37° C. After incubation, the cells were washed, fixed, permeabilized, and treated with a primary human antibody specific for SARS-CoV-2's spike protein (CR3022 epitope, Abcam, cat. no. ab273073) and a fluorescently (FITC) labeled secondary antibody (Sigma-Aldrich). Cellular uptake of SARS-CoV-2 was then examined with flow cytometry (Amnis® FlowSight® Imaging Flow Cytometer) and confocal microscopy (a Leica DMi8 microscope equipped with Aurox Clarity Laser Free Confocal Unit). To remove extracellularly attached viral particles, SARS-CoV-2-treated cells were trypsinized after incubation [Nakase et al. 2007 (Biochemistry (46) 492-501)]. Thus only internalized virus particles were measured with the flow cytometer. Our studies showed that SDCs increase the cellular uptake of SARS-CoV-2. Among SDC, lung-enriched SDC4 (see BioGPS gene expression database http://biogps.org) best facilitated cellular entry of SARS-CoV-2 (FIG. 1. Heat-inactivated SARS-CoV-2 uptake into K562 cells and SDC transfectants. FIG. 1A. Detected fluorescence intensities of SARS-CoV-2-treated cells were normalized to SARS-CoV-2-treated WT K562 cells as standards. The bars represent the mean±SEM of three independent experiments. Statistical significance vs. standards was assessed with analysis of variance (ANOVA). *p<0.05 vs. standards. FIG. 1B. Confocal microscopic visualization SARS-CoV-2 cellular entry into WT K562 cells and SDC transfectants. Representative images of three independent experiments are shown. Scale bar=10 μm.) Knockdown SDC4 expression with specific siRNA in SDC4 transfectants reduced cellular uptake of SARS-CoV-2 in SDC4 transfectants (FIG. 2. Changes in SDC4 expression levels and SARS-CoV-2 uptake following SDC4 knockdown. Detected SDC4 expression and SARS-CoV-2 uptake was normalized to those of SDC4 transfectants as standards. The bars represent the mean±SEM of three independent experiments. Statistical significance vs. standards was assessed with ANOVA. *p<0.05 vs. standards.) In the knockdown experiments, SDC4 transfectants were treated with SDC4 siRNA (100 nM siRNA, Santa Cruz Biotechnology, cat. no. sc-36588) according to the manufacturer's instructions. SDC4 expression was then measured with APC-labeled SDC4 antibodies (monoclonal rat IgG2A clone #336304, RnD Systems, Cat. no. FAB29181A) on a flow cytometer. SARS-CoV-2 uptake was examined according to the flow cytometry protocol described above.

Next, we investigated the ability to inhibit the uptake of heat-inactivated SARS-CoV-2 into SDC4 transfectants and WT K562 cells. Cells were co-incubated with 5 MOI of heat-inactivated SARS-CoV-2 for 18 h at 37° C. and the following SDC-targeting agents:
TAT peptide (amino acid sequence YGRKKRRQRRR, synthesized by PepScan) at a concentration of 25 μM;
penetratin (amino acid sequence RQIKIWFQNRRMKWKK, synthesized by PepScan) at a concentration of 25 μM;
polyarginine (R8, amino acid sequence RRRRRRRR, synthesized by PepScan) at a concentration of 25 μM;
monoclonal SDC4 antibody (monoclonal rat IgG2A clone #336304, RnD Systems, Cat. no. MAB29181) at 5 μg/ml;
recombinant SDC4 (RnD Systems, Cat. no. 2918-SD) at 2.5 μg/mL;
a peptide synthesized by combining conserved sections of the intracellular domain of SDC4 (RMKKKDEGEFYA) at a concentration of 25 μM;
SPRRARSV peptide (Pro681-Ser686, which includes the PRRAR heparin-binding motif) isolated from the S1 subunit of the SARS-CoV-2 spike protein at a concentration of 25 μM;
heparan sulfate (HS, Sigma-Aldrich, cat. no. H7640) at a concentration of 25 μg/ml;
Gö 6976 (Sigma-Aldrich, cat. no. 365250) at a concentration of 1 μM;
hexamethylene amiloride (HMA, Sigma-Aldrich, cat. no. A9561) at a concentration of 10 μM.

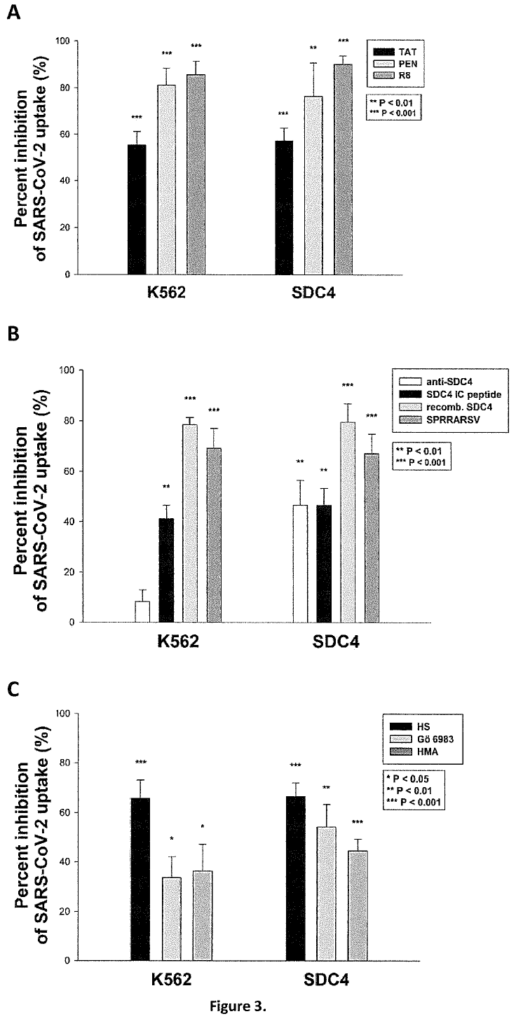

Flow cytometric studies showed that SDC-targeting agents significantly inhibited the uptake of heat-inactivated SARS-CoV-2 into both cell types, except for the SDC4 antibody that inhibited SARS-CoV-2 uptake only in WT K562 cells. (FIG. 3. Inhibition of SARS-CoV-2 cellular uptake with SDC-targeting agents in K562 cells and SDC4 transfectants. The effect of an inhibitor is expressed as percent inhibition, calculated with the following formula: [(X−Y)/X]×100, where X is the fluorescence intensity obtained on cells treated with SARS-CoV-2 in the absence of the inhibitor and Y is the fluorescence intensity obtained on cells treated with SARS-CoV-2 in the presence of inhibitor. The bars represent the mean±SEM of three independent experiments. Statistical significance vs. standards treated with SARS-CoV-2 in the absence of the inhibitor was assessed with ANOVA. *p<0.05 vs. standards; **p<0.01 vs. standards; ***p<0.001 vs. standards. FIG. 3A. TAT: Tat peptide; PEN: penetratin; R8: polyarginine; FIG. 3B. anti-SDC4: SDC4 antibody, SDC4 IC peptide: SDC4 intracellular domain peptide, recomb. SDC4: recombinant SDC4, FIG. 3C, HMA: hexamethylene amiloride.)

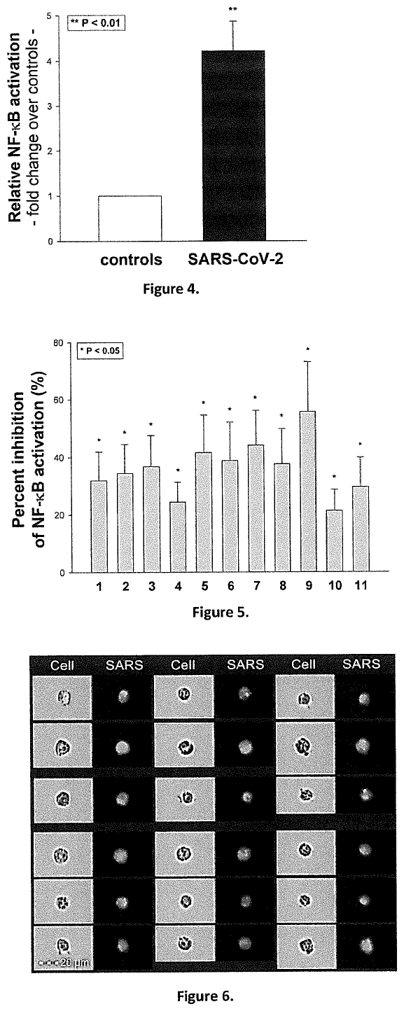

The SARS-CoV-2 virus induces a pronounced inflammatory response in the lung, which is ultimately responsible for the development of a cytokine storm leading to respiratory failure [Mason 2020 (Eur Respir J (55) 2000607)]. Therefore, inhibiting the inflammatory response-inducing effect of SARS-CoV-2 might be effective in preventing SARS-CoV-2-induced respiratory failure. A549/NF-κB-luc human lung epithelial cells were used to study SARS-CoV-2's inflammatory response inducing effect. As A549/NF-κB-luc cells are transfected with a plasmid containing a luciferase (luc) gene under the control of the inflammatory transcription factor NF-κB, the luciferase activity in these cells quantitatively indicates NF-κB activity [Letoha et al., 2006 (Mol Pharmacol. 69 (6): 2027-36)]. (Generation of the A549/NF-κB-luc cells: A549 human lung epithelial cells were transfected with plasmid pNF-κB-luc and pSV-2/neo plasmids—coding for firefly luciferase under the regulation of 5 NF-κB-responsive elements and the neo^{r} gene controlled by the SV40 enhancer/promoter—using Lipofectamine 2000 [Invitrogene™] transfection reagent. Following transfection, stable clones were selected with Geneticin G418 [400 mg/l]. The clones with the highest luciferase activity [i.e., NF-κB activity] were used to study SARS-CoV-2-induced NF-κB activity.) In our experiments, heat-inactivated SARS-CoV-2 significantly increased NF-κB activity. A549/NF-κB-luc cells plated on a 96-well luminoplate (Corning-Costar; Zenon Biotechnology Ltd., Szeged, Hungary) at a density of 3×10⁴ cells (in 200 μl medium)/well were treated with 5 MOI SARS-CoV-2 virus particles. After 18 h of incubation at 37° C., the cells were washed (with PBS), lysed (1× Bright-Glo Cell Culture Lysis reagent, 20-20 μl/well; Promega, Madison, Wis., USA) and after the addition of the luciferase substrate (Bright-Glo Luciferase Substrat, 20-20 μl/well; Promega) luminescence was measured with a luminometer (BioTek Cytation 3). FIG. 4 shows SARS-CoV-2-induced increase in NF-κB activity compared to untreated control cells. (FIG. 4. Heat-inactivated SARS-CoV-2 increases NF-κB activation in A549/NF-κB-luc cells. Detected luminescence intensities were normalized to A549/NF-κB-luc control cells untreated with SARS-CoV-2. The bars represent the mean±SEM of three independent experiments. Statistical significance vs. controls was assessed with ANOVA. **p<0.01 vs. standards.).

We then investigated the ability of the following agents co-administered with SARS-CoV-2 to inhibit SARS-CoV-2-induced NF-κB activation:
TAT peptide (amino acid sequence YGRKKRRQRRR, synthesized by PepScan) at a concentration of 25 μM (1);
penetratin (amino acid sequence RQIKIWFQNRRMKWKK, synthesized by PepScan) at a concentration of 25 μM (2);
polyarginine (R8, amino acid sequence RRRRRRRR, synthesized by PepScan) at a concentration of 25 μM (3);
SDC4 siRNA (Santa Cruz Biotech) at 100 nM (4);
monoclonal SDC4 antibody (monoclonal rat IgG2A clone #336304, RnD Systems, cat. no. MAB29181) at a concentration of 5 μg/mL (5);
recombinant SDC4 (RnD Systems, Cat. no. 2918-SD) at 2.5 μg/mL (6);
a peptide synthesized by combining conserved sections of the intracellular domain of SDC4 (RMKKKDEGEFYA) at a concentration of 25 μM (7);
SPRRARSV peptide (Ser680-Val687, which includes the PRRAR heparin-binding motif) isolated from the S1 subunit of the SARS-CoV-2 spike protein at a concentration of 25 μM (8);
heparan sulfate (HS, Sigma-Aldrich, cat. no. H7640) at a concentration of 25 μg/ml (9);
Gö 6976 (Sigma-Aldrich, cat. no, 365250) at a concentration of 1 μM (10);
hexamethylene amiloride (HMA, Sigma-Aldrich, cat. no. A9561) at a concentration of 10 μM (11).

FIG. 5. shows that the agents listed above significantly (p<0.05) inhibited NF-κB activation induced by 5 MOI of heat-inactivated SARS-CoV-2. Therefore, these agents are suitable for inhibiting the SARS-CoV-2-induced inflammatory response. (FIG. 5. Inhibition of SARS-CoV-2-induced NF-κB activation with SDC-targeting agents in A549/NF-κB-luc cells. The effect of an inhibitor is expressed as percent inhibition, calculated with the following formula: [(X−Y)/X]×100, where X is the luminescence intensity obtained on cells treated with SARS-CoV-2 in the absence of the inhibitor and Y is the luminescence intensity obtained on cells treated with SARS-CoV-2 in the presence of inhibitor. The bars represent the mean±SEM of three independent experiments. Statistical significance vs. standards treated with SARS-CoV-2 in the absence of the inhibitor was assessed with ANOVA. *p<0.05 vs. standards. 1: TAT peptide [TAT]; 2: penetratin [PEN]; 3: polyarginine [R8]; 4: SDC4 siRNA 5: anti-SDC4 antibody [anti-SDC4] 6: SDC4 intracellular domain peptide [SDC4 IC peptide] 7: recombinant SDC4 [recomb. SDC4] 8: SPRRARSV peptide 9: heparan sulfate [HS] 10: Gö 6983 11: hexamethylene amiloride [HMA]).

According to literature data, SARS-CoV-2 cannot infect mice due to the virus' inability to attach to the mouse ACE2 receptor [Zhou and Yang et al., 2020 (Nature (579): 270-273)]. in our uptake studies, RAW 264.7 murine macrophages internalized the heat-inactivated SARS-CoV-2 efficiently (FIG. 6. Cellular entry of SARS-CoV-2 into murine macrophages. WT K562 cells were incubated with 5 MOI heat-inactivated SARS-CoV-2 for 18 h at 37° C. After incubation, the cells were washed, fixed, permeabilized, and treated with a primary human antibody specific for SARS-CoV-2's spike protein [CR3022 epitope, Abeam, cat. no. ab273073] and a fluorescently [FITC] labeled secondary antibody [Sigma-Aldrich]. Cellular uptake of SARS-CoV-2 was then examined with imaging flow cytometry [Amnis® FlowSight® Imaging Flow Cytometer]. To remove extracellularly attached viral particles, SARS-CoV-2-treated cells were trypsinized after incubation. Cellular images of SARS-CoV-2 RAW 264.7 cells as detected with imaging flow cytometry. Representative images of three independent experiments are shown. Scale bar=20 μm.)

Then we investigated the ability of the following SDC-targeting agents co-administered with SARS-CoV-2 to inhibit SARS-CoV-2 uptake into RAW 264.7 murine macrophages:
SDC4 siRNA (100 nM, applied according to the manufacturer's protocol [Santa Cruz Biotech, cat. no. sc-36589]) (1);
anti-SDC4 antibody ([5G9], Santa Cruz Biotech, cat. no. sc-12766) at a concentration of 5 μg/mL (2);
recombinant SDC4 (RnD Systems, cat. no. 2918-SD) at 2.5 μg/mL (3);
a peptide synthesized by combining conserved sections of the intracellular domain of SDC4 (RMKKKDEGEFYA) at a concentration of 25 μM (4);
PRRAR peptide isolated from the heparin-binding motif of the S1 subunit of the SARS-CoV-2 spike protein (Pro681-Arg685) at a concentration of 25 μM (5);
heparan sulfate (HS, Sigma-Aldrich, cat. no. H7640) at a concentration of 25 μg/ml (6);
Gö 6976 (Sigma-Aldrich, cat, no. 365250) at a concentration of 1 μM (7);
hexamethylene amiloride (HMA, Sigma-Aldrich, cat. no. A9561) at a concentration of 10 μM (8).

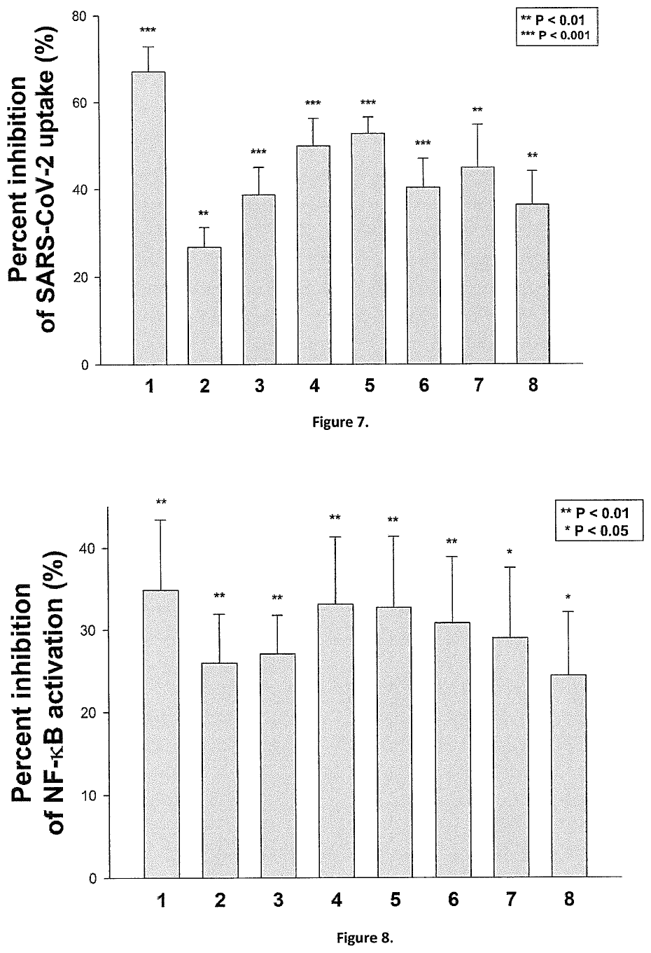

The listed SDC-targeting agents significantly (p<0.05) inhibited SARS-CoV-2 uptake into RAW 264/cells, highlighting the involvement of SDC4 in the cellular entry of SARS-CoV-2 in murine cells (FIG. 7. Inhibition of SARS-CoV-2 cellular uptake with SDC-targeting agents in RAW 264.7 transfectants. The effect of an inhibitor is expressed as percent inhibition, calculated with the following formula: [(X−Y)/X]×100, where X is the fluorescence intensity obtained on cells treated with SARS-CoV-2 in the absence of the inhibitor and Y is the fluorescence intensity obtained on cells treated with SARS-CoV-2 in the presence of inhibitor. The bars represent the mean±SEM of three independent experiments. Statistical significance vs. standards treated with SARS-CoV-2 in the absence of the inhibitor was assessed with ANOVA. **p<0.01 vs. standards; ***p<0.001 vs. standards. 1: SDC4 siRNA 2: anti-SDC4 antibody [anti-SDC4] 3: SDC4 intracellular domain peptide [SDC4 IC peptide] 4: recombinant SDC4 [recomb. SDC4] 5: PRRAR peptide 6: heparan sulfate [HS] 7: Gö 6983 8: hexamethylene amiloride [HMA].)

In RAW 264.7/NF-κB-luc cells, the same agents (i.e. SDC4 siRNA, anti-SDC4 antibody, SDC4 IC peptide, recombinant SDC4, SPRRAR peptide, HS, Gö 6983, HMA) also inhibited SARS-CoV-2-induced NF-κB activation (FIG. 8.). (Generation of RAW 264.7/NF-κB-luc cells: RAW 264.7 [5×10⁵/60 mm plate] subcultured the previous day were transformed overnight with the pNF-κB-luc and pSV-2/neo plasmids complexed with polyethylene-imine [jetPEI, Polyplus-Transfection®]. Geneticin-containing medium [400 mg/l] was used for selection. Clones showing the highest response to LPS activation [0.1 to 10 μg/mL, 6-10 h of incubation] were used for the assays). (FIG. 8. Inhibition of SARS-CoV-2-induced NF-κB activation with SDC-targeting agents in RAW 264.7/N F-κB-luc cells. RAW 264.7/NF-κB-luc cells plated on a 96-well luminoplate at a density of 3×10⁴ cells [in 200 μl medium]/well were treated with 5 MOI SARS-CoV-2 virus particles in the absence or presence of SDC-targeting inhibitors. After 18 h of incubation at 37° C., the cells were washed with PBS, lysed, and after the addition of the luciferase substrate, luminescence was measured with a luminometer [BioTek Cytation 3]. The effect of an inhibitor was expressed as percent inhibition, calculated with the following formula: [(X−Y)/X]×100, where X is the luminescence intensity obtained on cells treated with SARS-CoV-2 in the absence of the inhibitor and Y is the luminescence intensity obtained on cells treated with SARS-CoV-2 in the presence of inhibitor. The bars represent the mean±SEM of three independent experiments. Statistical significance vs. standards treated with SARS-CoV-2 in the absence of the inhibitor was assessed with ANOVA. *p<0.05 vs. standards. 1: SDC4 siRNA 2: anti-SDC4 antibody [anti-SDC4] 3: SDC4 intracellular domain peptide [SDC4 IC peptide] 4: recombinant SDC4 [recomb. SDC4] 5: PRRAR peptide 6: heparan sulfate [HS] 7: Gö 6983 8: hexamethylene amiloride [HMA].)

The present invention is based on the surprising finding that specific peptides, antibodies, macromolecular ligands, or low molecular weight agents targeting or derived from SDCs with the ability to inhibit SDC-dependent endocytosis are also able to inhibit the cellular uptake of SARS-CoV-2, therefore, these agents are suitable for inhibiting SARS-CoV-2 infection and reducing the SARS-CoV-2-induced inflammatory response.

The present invention relates to agents that inhibit the binding of SARS-CoV-2 to SDCs or the endocytosis of SARS-CoV-2 with SDCs for use in the treatment of SARS-CoV-2 infection by inhibiting the cellular entry of SARS-CoV-2 and/or the SARS-CoV-2-induced inflammatory response.

Agents which can be used according to the invention are:

a.) an SDC-specific mono- or polyclonal antibody, an antibody specific for the human SDC isoforms (SDC1, -2, -3, -4); or b.) SDC-specific peptides, peptide ligands that interact with the extracellular domain of human SDC isoforms (SDC1, -2, -3, -4), such as TAT (amino acid sequence YGRKKRRQRRR), penetratin (amino acid sequence RQIKIWFQNRRMKW), polyarginine (amino acid sequence RRRRRRRR) and their analogs of at least 4 amino acids in length, synthesized from D-, L- or other amino acid derivatives, or peptides interacting with the glycosaminoglycan side chains of SDCs, containing the conserved heparin-binding motif, PRRAR, or analogs of at least 4 amino acids in length, synthesized from D-, L-, or other amino acid derivatives or conjugates of the former with other active agents and carriers (liposomes and other polymeric nanoparticles); or

b.) SDC-specific low- and high-molecular-weight ligands, any agents capable of binding to the extra- and intracellular domains of SDC, including the glycosaminoglycan side chains of SDCs; or

c.) agents derived from SDCs, recombinant proteins derived from the extra- and intracellular domains of human SDC isoforms (SDC1, -2, -3, -4) and analogs synthesized from D-, L- or other amino acid derivatives of at least four amino acids in length, and derivatives of glycosaminoglycan side chains and glycosaminoglycan analogs of human SDC isoforms (SDC1, -2, -3, -4); or

d.) agents reducing the expression of SDCs, nucleic acid-based agents capable of reducing the expression of SDCs and their conjugates and complexes packaged in viral and non-viral carriers (liposomes and other polymeric nanoparticles); or

e.) agents containing the conserved intracellular domain of SDCs, peptides, peptidomimetics, and recombinant proteins of at least four amino acids in length, synthesized from D-, L- or other amino acid derivatives containing the sequence of any conserved region of the intracellular domain of human SDC isoforms (SDC1, -2, -3, -4); or

f.) an inhibitor of endocytosis of SDCs, low molecular weight agents that inhibit the interaction of SDCs with PKCα, the RAC1 and Wnt pathways.

The present invention also relates to a method of inhibiting SARS-CoV-2 cellular entry and SARS-CoV-2-induced inflammatory response with agents able to block SARS-CoV-2's attachment to SDCs or the SDC-mediated cellular uptake of SARS-CoV-2 via attaching the agent to the target cell of SARS-CoV-2.

In view of the above, the present invention provides a method of inhibiting the cellular entry of SARS-CoV-2 and the SARS-CoV-2-induced inflammatory response by preventing SARS-CoV-2 from binding to cell surface SDCs or entering the cells via SDCs, by means of:

a) attaching a mono- or polyclonal antibody, peptide or SDC-specific macromolecular ligand or SDC derivative specific for the extra- or intracellular domains of SDC to the SDC-expressing target cells of SARS-CoV-2, thereby preventing SARS-CoV-2 from binding to and entering the target cell and inducing an inflammatory response; or

b) delivering an SDC expression reducing agent into the SARS-CoV-2 target cells to prevent the cellular entry of SARS-CoV-2 and the SARS-CoV-2-induced inflammatory response by inhibiting SDC expression; or

c) delivering a peptide containing the conserved intracellular domain of SDCs into SARS-CoV-2 target cells to competently inhibit SDC-dependent signaling pathways and prevent SARS-CoV-2 from entering the cell via SDCs and inducing an inflammatory response; or

d) delivering an agent that inhibits SDC endocytosis to SARS-CoV-2 target cells to prevent the cellular entry of SARS-CoV-2 by inhibiting SDC-mediated endocytosis and the SARS-CoV-2-induced inflammatory response; or

e) delivering an agent that inhibits SDC-mediated signaling pathways to SARS-CoV-2 target cells to prevent the cellular entry of SARS-CoV-2 and SARS-CoV-2-induced inflammatory response by inhibiting SDC-mediated endocytosis; or

f) attaching drugs from the extracellular domain of the SDC, such as a peptide, peptidomimetic, protein, glycosaminoglycan or heparin derivative to SARS-CoV-2, thereby preventing SARS-CoV-2 from entering the target cell and inducing and inflammatory response.

Attachment can be achieved via systemic or oral administration or inhalation.

Delivery can be achieved via systemic or oral administration or inhalation.

As SDC-specific mono- or polyclonal antibodies the following agents can be used:
antibodies specific for human SDC isoforms (SDC1, -2, -3, -4), or
as an SDC-specific peptide, peptide ligand that interacts with the extracellular domain of human SDC isoforms (SDC1, -2, -3, -4), such as TAT (amino acid sequence YGRKKRRQRRR), penetratin (amino acid sequence RQIKIWFQNRRMKW), polyarginine (amino acid sequence RRRRRRRR) and their analogs of at least four amino acids in length, synthesized from D-, L- or other amino acid derivatives, or peptides interacting with the glycosaminoglycan side chains of SDCs—containing the conserved heparin-binding motif PRRAR—of at least four amino acids in length, or their analogs synthesized from D-, L- or other amino acid derivatives or conjugates of the former with other active agents and carriers (liposomes and other polymeric nanoparticles);
as SDC specific low- and high-molecular-weight ligands, any agents capable of binding to the extra- and intracellular domains of SDC, including the glycosaminoglycan side chains of SDCs;
as agents derived from SDCs, recombinant proteins derived from the extra- and intracellular domains of human SDC isoforms (SDC1, -2, -3, -4) and analogs synthesized from D-, L- or other amino acid derivatives of at least four amino acids in length, and derivatives of glycosaminoglycan side chains and glycosaminoglycan analogs of human SDC isoforms (SDC1, -2, -3, -4);
as agents reducing the expression of SDCs, nucleic acid-based drugs capable of reducing the expression of SDCs and their conjugates or complexes packaged in viral and non-viral carriers (liposomes and other polymeric nanoparticles);
as agents containing the conserved intracellular domain of SDCs, peptides, peptidomimetics, and recombinant proteins of at least four amino acids in length, synthesized from D-, L- or other amino acid derivatives and containing the sequence of any conserved region of the intracellular domain of human SDC isoforms (SDC1, -2, -3, -4);
as an inhibitor of endocytosis of SDCs, low molecular weight agents inhibiting the interaction of SDCs with PKCα, the RAC1 and Wnt pathways.

A further embodiment of the invention is the use of an agent that inhibits the binding of SARS-CoV-2 to SDCs or the endocytosis of SARS-CoV-2 with SDCs as a therapeutic agent for treating SARS-CoV-2 infection.

1-7. (canceled)

8. Agents, selected from the groups
a. anti-syndecan antibodies;
b. TAT (amino acid sequence YGRKKRRQRRR), penetratin (amino acid sequence RQIKIWFQNRRM;KW), polyarginine (amino acid sequence RRRRRRRR) and their analogs of at least four amino acids in length, synthesized from D-, L- or other amino acid derivatives, or peptides and peptidomimetics interacting with the glycosaminoglycan side chains of SDCs, containing the conserved heparin-binding motif, PRRAR, or analogs of at least four amino acids in length, synthesized from D-, L- or other amino acid derivatives or conjugates of the former with other active agents and carriers (liposomes and other polymeric nanoparticles);
c. heparin-binding peptides, such as PRRAR derived from fibronectin;
d. RMKKKDEGEFYA motif synthesized by combining conserved sections of the intracellular domain of SDC4;
e. syndecan-4 siRNA;
f. Gö 6976, hexamethylene-amiloride and heparin derivatives,
that inhibit the binding of SARS-CoV-2 to SDCs or the endocytosis of SARS-CoV-2 with syndecans (SDCs) for the use in the treatment of SARS-CoV-2 infection by inhibiting the cellular entry of SARS-CoV-2 and/or the SARS-CoV-2-induced inflammatory response.

9. Agents according to claim 8, selected from the groups
a. an antibody specific for the extracellular domains (residues 1-252 [SDC1], residues 1-143 [SDC2]; residues 1 to 384 [SDC3] and residues 1-140 [SDC4]) of SDC isoforms as anti-syndecan antibodies;
b. hexamethylene-amiloride and its derivative as inhibitor of SDC-mediated endocytosis.

10. A method of inhibiting SARS-CoV-2 infection, the cellular entry of SARS-CoV-2, and the SARS-CoV-2-induced inflammatory response, comprising attaching an agent selected from
a. anti-syndecan antibodies;
b. TAT (amino acid sequence YGRKKRRQRRR), penetratin (amino acid sequence RQIKIWFQNRRM;KW), polyarginine (amino acid sequence RRRRRRRR) and their analogs of at least four amino acids in length, synthesized from D-, L- or other amino acid derivatives, or peptides and peptidomimetics interacting with the glycosaminoglycan side chains of SDCs, containing the conserved heparin-binding motif, PRRAR, or analogs of at least four amino acids in length, synthesized from D-, L. or other amino acid derivatives or conjugates of the former with other active agents and carriers;
c. D-, L- or other amino acid derivatives or conjugates of the former with other active agents and carriers (liposomes and other polymeric nanoparticles);
d. heparin-binding peptides, such as PRRAR derived from fibronectin;
e. RMKKKDEGEFYA motif synthesized by combining conserved sections of the intracellular domain of SDC4;
f. syndecan-4 siRNA;
g. Gö 6976, hexamethylene-amiloride,
that inhibit the binding of SARS-CoV-2 to SDC or the SDC-mediated endocytosis of SARS-CoV-2 to the target cells of SARS-CoV-2.

11. The method of claim 10, comprising attaching
a. the SDC-specific antibody, to the SARS-CoV-2 target cells expressing the SDCs,
b. attaching glycosaminoglycans from the extracellular domain of SDCs to SARS-CoV-2.

12. The method of claim 10, wherein
a. as SDC specific antibody an antibody specific for the extracellular domains (residues 1-252 [SDC1], residues 1-143 [SDC2]; residues 1 to 384 [SDC3] and residues 1-140 [SDC4]) of SDC isoforms;
b. as endocytosis inhibitor hexamethylene-amiloride or its derivatives
is used.

13. A method of treating SARS-CoV-2 infection which comprises administering to a patient in need thereof an agent selected from the groups
a. anti-syndecan antibodies;
b. TAT (amino acid sequence YGRKKRRQRRR), penetratin (amino acid sequence RQIKIWFQNRRM;KW), polyarginine (amino acid sequence RRRRRRRR) and their analogs of at least four amino acids in length, synthesized from D-, L- or other amino acid derivatives, or peptides and peptidomimetics interacting with the glycosaminoglycan side chains of SDCs, containing the conserved heparin-binding motif, PRRAR, or analogs of at least four amino acids in length, synthesized from D-, L- or other amino acid derivatives or conjugates of the former with other active agents and carriers (liposomes and other polymeric nanoparticles);
c. heparin-binding peptides, such as PRRAR derived from fibronectin;
d. RMKKKDEGEFYA motif synthesized by combining conserved sections of the intracellular domain of SDC4;
e. syndecan-4 siRNA;
f. Gö 6976 and hexamethylene-amiloride, that inhibit the binding of SARS-CoV-2 to SDCs or the endocytosis of SARS-CoV-2 with SDCs.

14. The method according to claim 13, wherein the agent is selected from
a. an antibody specific for the extracellular domains (residues 1-252 [SDC1], residues 1-143 [SDC2]; residues 1 to 384 [SDC3] and residues 1-140 [SDC4]) of SDC isoforms as anti-syndecan antibodies;
b. hexamethylene-amiloride and its derivatives as inhibitors of SDC-mediated endocytosis of SARS-CoV-2.

15. The method of claim 11, wherein
a. the SDC specific antibody is an antibody specific for the extracellular domains (residues 1-252 [SDC1], residues 1-143 [SDC2]; residues 1 to 384 [SDC3] and residues 1-140 [SDC4]) of SDC isoforms; and
b. the endocytosis inhibitor is hexamethylene-amiloride or its derivatives.
